(19) United States
(12) Patent Application Publication
(10) Pub. No.: US 2010/0047876 A1
(43) Pub. Date: Feb. 25, 2010

(54) HIERARCHICAL ASSEMBLY OF POLYNUCLEOTIDES

(75) Inventor: George M. Church, Brookline, MA (US)

Correspondence Address: BANNER & WITCOFF, LTD., 28 STATE STREET, SUITE 1800, BOSTON, MA, 02109-1701, US

(73) Assignee: President and Fellows of Harvard College, Cambridge, MA (US)

(21) Appl. No.: 12/533,141

(22) Filed: Jul. 31, 2009

Related U.S. Application Data
(60) Provisional application No. 61/087,357, filed on Aug. 8, 2008.

(51) Int. Cl. C12P 19/34

(52) U.S. Cl. 435/91.2

(57) ABSTRACT

Methods, compositions and apparatuses for hierarchical assembly of oligonucleotide sequences are provided.

### RELATED APPLICATION

This application claims priority to U.S. Provisional Patent Application No. 61/087,357, filed on Aug. 8, 2008 is hereby incorporated herein by reference in its entirety for all purposes.

### STATEMENT OF GOVERNMENT INTERESTS

This invention was made with Government support under DE-FG02-03ER63445 awarded by the Department of Energy. The Government has certain rights in the invention.

### FIELD

The present invention relates to novel methods, compositions and apparatuses for making polynucleotide sequences.

### BACKGROUND

In order to lower costs of enzymatic assembly of large DNAs from smaller, chemically-synthesized oligonucleotides, oligonucleotide chips are typically used (Tian et al. (2004) Nature 432:1050). The subsequent release of massive numbers of oligonucleotides into a small number of pools, however, results in considerable crosstalk during annealing, as well as during ligase or polymerase, assembly reactions.

### SUMMARY

The present invention is based in part on the surprising discovery of a new method to hierarchically assemble nucleic acid sequences (e.g., DNA sequences) using oligonucleotide arrays (e.g., oligonucleotide chips).

In certain exemplary embodiments, a method of making a polynucleotide is provided. The method includes the steps of providing an oligonucleotide array having a plurality of adjacent, discrete features attached thereto wherein each feature comprises a substrate oligonucleotide, contacting a first discrete feature having a first substrate attached thereto with an oligonucleotide primer (or primers), allowing the oligonucleotide primer to hybridize to the first substrate oligonucleotide and extending the substrate oligonucleotide to generate an extended oligonucleotide, releasing the extended oligonucleotide and allowing the extended oligonucleotide to contact (e.g., by diffusion) an adjacent, second discrete feature having a second substrate attached thereto, and allowing the extended oligonucleotide to hybridize to the second substrate oligonucleotide and extending the hybridized extended oligonucleotide and second substrate oligonucleotide to generate a first polynucleotide.

In certain aspects, the step of releasing is performed by contacting the extended oligonucleotide with a helicase, a strand displacement polymerase or heat. In other aspects, the oligonucleotide array includes a chip, a slide or a plate. In certain aspects, amplification is performed by polymerase chain reaction or ligase chain reaction. In still other aspects, comprising removing one or both of an extended oligonucleotide and a first polynucleotide having a mismatch, e.g., using one or more of mismatch-sensitive hybridization, mutS binding, MutHSL cleavage near the mismatch and cleavage at the mismatch. In certain aspects, the oligonucleotide primer is between 8 and 25 nucleotides in length. In other aspects, the first and second substrate oligonucleotides are between 50 and 100 nucleotides in length. In yet other aspects, the first polynucleotide is greater than 100 nucleotides in length or between 100 and 150 nucleotides in length. In other aspects, the primer(s) are added by ink-jet printing.

In certain aspects, the method further includes the steps of releasing the first polynucleotide and allowing the first polynucleotide to contact an adjacent, third discrete feature having a third substrate attached thereto, and allowing the first polynucleotide to hybridize to the third substrate oligonucleotide and extending the hybridized first polynucleotide and third substrate oligonucleotide to generate a second polynucleotide. In certain aspects, the second polynucleotide is greater than 200 nucleotides in length or between 200 and 300 nucleotides in length.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIG. 1 schematically depicts an oligonucleotide chip having a square grid.

FIG. 2 schematically depicts an oligonucleotide chip having a checkerboard grid.

### DETAILED DESCRIPTION

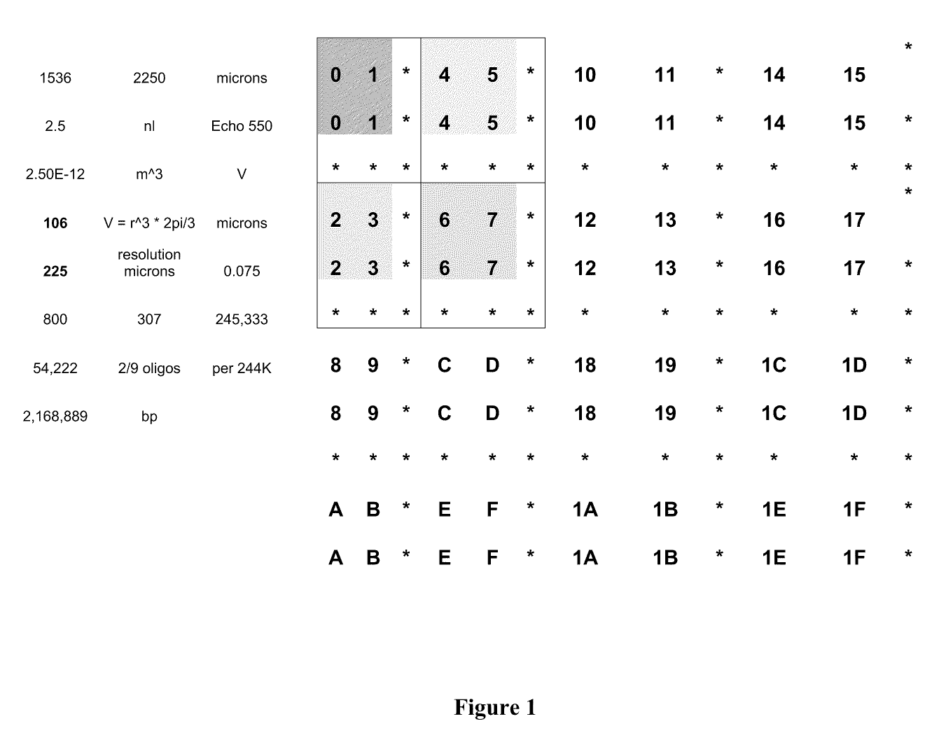

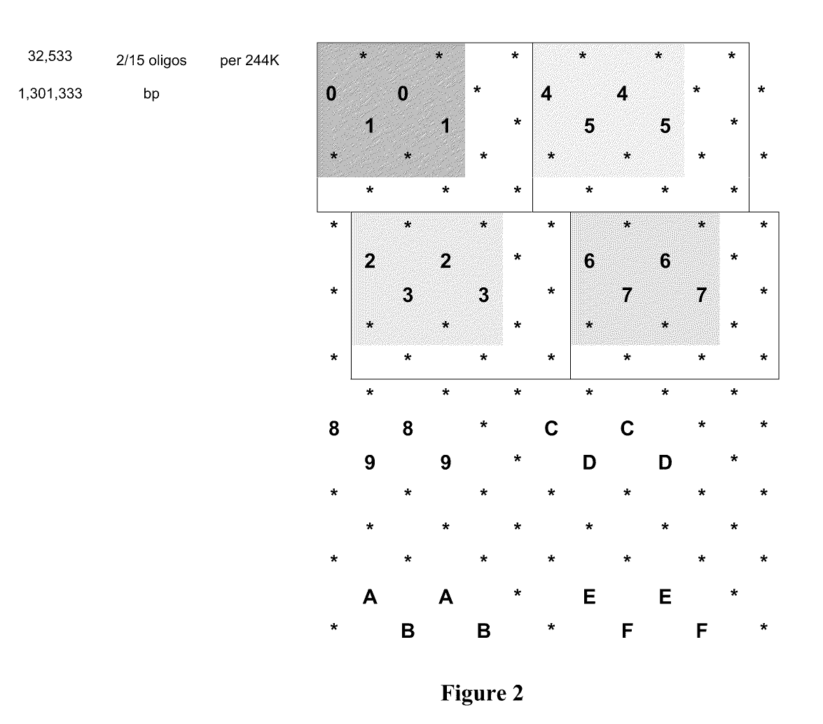

The principles of the present invention are based in part of the discovery of methods and compositions for hierarchically assembling oligonucleotide and/or polynucleotide sequences using a support (e.g., an oligonucleotide (e.g., DNA) array). The support is physically designed such that successive synthesis (e.g., intermediate) reactions and successive assembly (e.g., final) reactions are performed in physically adjacent regions on the support (e.g., oligonucleotide array) (e.g., such that pairs join up first then pairs of pairs, and the like (as depicted in FIGS. 1 and 2)).

In certain exemplary embodiments, a primer (e.g., a universal or quasi-universal primer (e.g., a 10-mer)) that binds to a substrate oligonucleotide (e.g., a 60-mer) is hybridized to the substrate oligonucleotide and the substrate oligonucleotide is extended (e.g., in presence of a polymerase, Mg-buffer, and dNTPs). The extended substrate oligonucleotide can then be released (e.g., by helicase, strand-displacement-polymerase or heat) and allowed to contact (e.g., by diffusion) and hybridize to a second substrate oligonucleotide (e.g., a 60-mer) having at least a portion of complementarity to the extended substrate oligonucleotide. The extended substrate oligonucleotide can hybridize to a complementary region (e.g., a 10 base pair region) of the second substrate oligonucleotide (e.g., 75 microns away an adjacent chip region, e.g., near the 3′ end of that oligonucleotide) and the hybridized, extended substrate oligonucleotide and second substrate oligonucleotide can be extended to form a first polynucleotide. A properly extended first polynucleotide would be 110 base pairs long. At this point the first polynucleotide could be amplified, or extended on a third substrate oligonucleotide (e.g., a 60-mer), or they can bind to each other 110-mers by e.g. a 10 bp region and then extend (and/or amplify) producing 210-mers.

In certain exemplary embodiments, alternative methods of priming can be used. Such methods include, but are not limited to, the use of dendrimers, 5′ immobilized primers, and/or panhandle primers to improve initial or subsequent priming to control diffusion. In certain aspects, reactions can optionally be washed in between steps under non-denaturing conditions and/or can optionally be washed under denaturing conditions (e.g., in the presence of formamide and/or heat or the like). In certain aspects, washing steps can optionally be followed by partial or complete drying, optionally employing strategic surface chemistry like non-wettable regions between oligonucleotide spots on the support. In certain exemplary embodiments, the sequence layout strategy can aim to minimize consequences of droplet splatter or misalignment by recognizing that each original oligonucleotide pair is surrounded by eight other pairs. For example in FIG. 1, the pair 6-7 is surround by 0-1, 2-3, 4-5, 8-9, 10-11, 12-13, 18-19, and C-D. Each oligonucleotide type of each pair can have different quasi-universal tags at its 3′ end of. The oligonucleotide can then be reused one grid-point removed in each direction.

Ink-jet printing (e.g., Echo 550 (Worldwide Website: bucher.ch/en/products/Labcyte/Echo-550-Acoustic-Liquid-Handler.html)) of aqueous enzyme(s) and/or substrate (e.g., primer and/or substrate oligonucleotides) mix in small (e.g., approximately 2.5 nanoliter) droplets can be used in the methods described herein (e.g., the $500 Agilent 244K 60-mer chips (Worldwide Website: chem.agilent.com/scripts/pds.asp?1page=36199), Nimblegen, Febit, or Combimatrix)). Since commercially available ink-jet printers (e.g., such as the Echo) can operate from 384-well plate, this strategy could easily be extended to a number of primer types.

In certain exemplary embodiments, one or more oligonucleotide and/or polynucleotide sequences described herein are immobilized on a support (e.g., a solid and/or semi-solid support). The support can be simple square grids, checkerboard (e.g., offset) grids, hexagonal arrays and the like. Suitable supports include, but are not limited to, slides, beads, chips, particles, strands, gels, sheets, tubing, spheres, containers, capillaries, pads, slices, films, plates and the like. In various embodiments, a solid support may be biological, nonbiological, organic, inorganic, or any combination thereof.

When using a support that is substantially planar, the support may be physically separated into regions (e.g., discrete features), for example, with trenches, grooves, wells, or chemical barriers (e.g., hydrophobic coatings, etc.). In certain exemplary embodiments, physically separate regions (e.g., discrete features) are absent or are easily removable such that an oligonucleotide and/or polynucleotide at one discrete feature can contact an oligonucleotide and/or polynucleotide at an adjacent discrete feature. The current minimum drop size for apparatuses such as the Echo 550 & 555 is 2.5 nl, which corresponds to a 106 micron radius hemisphere. However, the minimum drop size for ink jet printing is more generally considerably less than that. In certain exemplary embodiments, redundant adjacent printed oligonucleotides can be used to handle large and/or imprecisely placed drops. In other exemplary embodiments, multiple drops can be used to handle relatively coarse oligonucleotide arrays.

In certain exemplary embodiments, a support is an oligonucleotide array such as, e.g., a microarray. As used herein, the terms “oligonucleotide array” and “microarray” refer in one embodiment to a type of assay that comprises a solid phase support having a substantially planar surface on which there is an array of spatially defined non-overlapping regions or sites that each contain an immobilized hybridization probe. “Substantially planar” means that features or objects of interest, such as probe sites, on a surface may occupy a volume that extends above or below a surface and whose dimensions are small relative to the dimensions of the surface. For example, beads disposed on the face of a fiber optic bundle create a substantially planar surface of probe sites, or oligonucleotides disposed or synthesized on a porous planar substrate creates a substantially planar surface. Spatially defined sites may additionally be “addressable” in that its location and the identity of the immobilized probe at that location are known or determinable.

Oligonucleotides and/or polynucleotides immobilized on microarrays include nucleic acids that are generated in or from an assay reaction. Typically, the oligonucleotides and/or polynucleotides on microarrays are single stranded and are covalently attached to the solid phase support, usually by a 5′-end or a 3′-end. The density of non-overlapping regions containing nucleic acids in a microarray is typically greater than 100 per cm², and more typically, greater than 1000 per cm². Microarray technology is reviewed in the following exemplary references: Schena, Editor, Microarrays: A Practical Approach (IRL Press, Oxford, 2000); Southern, Current Opin. Chem. Biol., 2: 404-410 (1998); Nature Genetics Supplement, 21:1-60 (1999); and Fodor et al, U.S. Pat. Nos. 5,424,186; 5,445,934; and 5,744,305.

Methods of immobilizing oligonucleotides to a support are described are known in the art (beads: Dressman et al. (2003) Proc. Natl. Acad. Sci. USA 100:8817, Brenner et al. (2000) Nat. Biotech. 18:630, Albretsen et al. (1990) Anal. Biochem. 189:40, and Lang et al. Nucleic Acids Res. (1988) 16:10861; nitrocellulose: Ranki et al. (1983) Gene 21:77; cellulose: (Goldkorn (1986) Nucleic Acids Res. 14:9171; polystyrene: Ruth et al. (1987) Conference of Therapeutic and Diagnostic Applications of Synthetic Nucleic Acids, Cambridge U.K.; Teflon-acrylamide: Duncan et al. (1988) Anal. Biochem. 169:104; polypropylene: Polsky-Cynkin et al. (1985) Clin. Chem. 31:1438; nylon: Van Ness et al. (1991) Nucleic Acids Res. 19:3345; agarose: Polsky-Cynkin et al., Clin. Chem. (1985) 31:1438; and sephacryl: Langdale et al. (1985) Gene 36:201; latex: Wolf et al. (1987) Nucleic Acids Res. 15:2911).

As used herein, the term “attach” refers to both covalent interactions and noncovalent interactions. A covalent interaction is a chemical linkage between two atoms or radicals formed by the sharing of a pair of electrons (i.e., a single bond), two pairs of electrons (i.e., a double bond) or three pairs of electrons (i.e., a triple bond). Covalent interactions are also known in the art as electron pair interactions or electron pair bonds. Noncovalent interactions include, but are not limited to, van der Waals interactions, hydrogen bonds, weak chemical bonds (i.e., via short-range noncovalent forces), hydrophobic interactions, ionic bonds and the like. A review of noncovalent interactions can be found in Alberts et al., in Molecular Biology of the Cell, 3d edition, Garland Publishing, 1994.

In certain exemplary embodiments, methods of isolating oligonucleotides and/or polynucleotides include, but are not limited to any combinations of: soft or hard lithography microfluidics (e.g. polydimethylsiloxane (PDMS) or Xeotron/Atatic (Tian et al., supra)) boundaries; photolithographic construction and/or destruction of impermeant barriers; gel boundaries; gel embedding (Worldwide Website biohelix.com/technology.asp) or the like.

In certain exemplary embodiments, the assembly products (e.g., oligonucleotides and/or polynucleotides) of one or more of the methods described herein can be amplified from single molecules using e.g., polymerase and/or ligase chain reactions, thermal cycling or isothermally using zero, one or two optionally immobilized specific or general primers or no primers at all (e.g., for primase-based whole genome amplification (PWGA)). Resulting polymerase colonies (polonies) can then be sequenced. Polonies which have the incorrect sequence can be selectively destroyed or released, e.g. via photo-caged nitrobenzyl linkages, or the correct polonies can be released by similar means into a captured flow.

Amplification methods may comprise contacting an oligonucleotide and/or polynucleotide with one or more primers that specifically hybridize to the nucleic acid under conditions that facilitate hybridization and chain extension. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, isothermal amplification (e.g., rolling circle amplification (RCA), hyperbranched rolling circle amplification (HRCA), strand displacement amplification (SDA), helicase-dependent amplification (HDA), PWGA) or any other nucleic acid amplification method using techniques well known to those of skill in the art. polymerase and/or ligase chain reactions. thermal cycling (PCR) or isothermally (e.g. RCA, hRCA, SDA, HDA, PWGA (Worldwide Website: biohelix.com/technology.asp)).

In certain exemplary embodiments, methods of determining the nucleic acid sequence of one or more oligonucleotides and/or polynucleotides are provided. Determination of the nucleic acid sequence of an oligonucleotide and/or polynucleotide can be performed using variety of sequencing methods known in the art including, but not limited to, ‘next generation’ sequencing methods such as, e.g., polymerase methods using fluorescent-dNTPs (Mitra et al. (2003) Analyt. Biochem. 320:55-65) or ligase methods using 5-mers to 9-mers (Shendure et al. (2005) Science 309(5741):1728), massively parallel signature sequencing (MPSS), sequencing by hybridization (SBH) and the like, sequencing by ligation (SBL), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. A variety of light-based sequencing technologies are known in the art (Landegren et al. (1998) Genome Res. 8:769-76; Kwok (2000) Pharmocogenomics 1:95-100; and Shi (2001) Clin. Chem. 47:164-172).

In certain exemplary embodiments, the methods described herein include one or more strategies for error correction in the oligonucleotides and/or polynucleotides described herein. Error correction methods include (but are not limited to): mismatch-sensitive hybridization (Tian et al., supra); mutS binding (Carr wet al. (2004) Nucleic Acids Res. 32(20):e162); MutHSL cleavage near mismatches (Smith et al. (1997) Proc. Natl. Acad. Sci. USA 94(13):6847); and cleavage directly at mismatches (Bang and Church (2008) Nat. Methods. 5(1):37-9.). Error correction can be performed by adding droplets containing components of one or more error correction methods described herein.

Proteins involved in mismatch repair, such as mismatch binding proteins, can be used to select oligonucleotides and/or polynucleotides having the correct nucleotide sequence. Mismatch repair proteins bind to a variety of DNA mismatches, deletions and insertions (Carr et al. (2004) Nucleic Acids Res. 32:e162). Accordingly, mismatch binding proteins can be used to bind to oligonucleotides and/or polynucleotides sequences which have errors. Double-stranded oligonucleotides and/or polynucleotides sequences that are error free may then be separated from double-stranded oligonucleotides sequences bound to mismatch binding proteins. Thus, error-free oligonucleotides and/or polynucleotides sequences can be effectively separated from oligonucleotide sequences that contain errors.

The term “DNA repair” refers to a process wherein sequence errors in a nucleic acid (DNA:DNA duplexes, DNA:RNA and, for purposes herein, also RNA:RNA duplexes) are recognized by a nuclease that excises the damaged or mutated region from the nucleic acid; and then further enzymes or enzymatic activities synthesize a replacement portion of a strand(s) to produce the correct sequence.

The term “DNA repair enzyme” refers to one or more enzymes that correct errors in nucleic acid structure and sequence, i.e., recognizes, binds and corrects abnormal base-pairing in a nucleic acid duplex. Examples of DNA repair enzymes include, but are not limited to, proteins such as mutH, mutL, mutM, mutS, mutY, dam, thymidine DNA glycosylase (TDG), uracil DNA glycosylase, AlkA, MLH1, MSH2, MSH3, MSH6, Exonuclease I, T4 endonuclease V, Exonuclease V, RecJ exonuclease, FEN1 (RAD27), dnaQ (mutD), polC (dnaE), or combinations thereof, as well as homologs, orthologs, paralogs, variants, or fragments of the forgoing. Enzymatic systems capable of recognition and correction of base pairing errors within the DNA helix have been demonstrated in bacteria, fungi and mammalian cells. and the like.

As used herein the terms “mismatch binding agent” or “MMBA” refer to an agent that binds to a double stranded nucleic acid molecule that contains a mismatch. The agent may be chemical or proteinaceous. In certain embodiments, an MMBA is a mismatch binding protein (MMBP) such as, for example, Fok I, MutS, T7 endonuclease, a DNA repair enzyme as described herein, a mutant DNA repair enzyme as described in U.S. Patent Publication No. 2004/0014083, or fragments or fusions thereof. Mismatches that may be recognized by an MMBA include, for example, one or more nucleotide insertions or deletions, or improper base pairing, such as A:A, A:C, A:G, C:C, C:T, G:G, G:T, T:T, C:U, G:U, T:U, U:U, 5-formyluracil (fU):G, 7,8-dihydro-8-oxo-guanine (8-oxoG):C, 8-oxoG:A or the complements thereof.

As used herein, the terms “MLH1” and “PMS1” (PMS2 in humans) refers to the components of the eukaryotic mutL-related protein complex, e.g., MLH1-PMS1, that interacts with MSH2-containing complexes bound to mispaired bases. Exemplary MLH1 proteins include, for example, polypeptides encoded by nucleic acids having the following GenBank accession Nos. AI389544 (D. melanogaster), AI387992 (D. melanogaster), AF068257 (D. melanogaster), U80054 (Rattus norvegicus) and U07187 (S. cerevisiae), as well as homologs, orthologs, paralogs, variants, or fragments thereof.

As used herein, the term “MSH2” refers to a component of the eukaryotic DNA repair complex that recognizes base mismatches and insertion or deletion of up to 12 bases. MSH2 forms heterodimers with MSH3 or MSH6. MSH2 proteins include, for example, polypeptides encoded by nucleic acids having the following GenBank accession Nos.: AF109243 (A. thaliana), AF030634 (Neurospora crassa), AF002706 (A. thaliana), AF026549 (A. thaliana), L47582 (H. sapiens), L47583 (H. sapiens), L47581 (H. sapiens) and M84170 (S. cerevisiae) and homologs, orthologs, paralogs, variants, or fragments thereof. MSH3 proteins include, for example, polypeptides encoded by the nucleic acids having GenBank accession Nos.: J04810 (H. sapiens) and M96250 (Saccharomyces cerevisiae) and homologs, orthologs, paralogs, variants, or fragments thereof. MSH6 proteins include, for example, polypeptides encoded by nucleic acids having the following GenBank accession Nos.: U54777 (H. sapiens) and AF031087 (M. musculus) and homologs, orthologs, paralogs, variants, or fragments thereof.

As used herein, the term “mutH” refers to a latent endonuclease that incises the unmethylated strand of a hemimethylated DNA, or makes a double strand cleavage on unmethylated DNA, 5′ to the G of d(GATC) sequences. The term is meant to include prokaryotic mutH (e.g., Welsh et al., 262 J. Biol. Chem. 15624 (1987)) as well as homologs, orthologs, paralogs, variants, or fragments thereof.

As used herein, the term “mutHLS” refers to a complex between mutH, mutL, and mutS proteins (or homologs, orthologs, paralogs, variants, or fragments thereof).

As used herein, the term “mutL” refers to a protein that couples abnormal base-pairing recognition by mutS to mutH incision at the 5′-GATC-3′ sequences in an ATP-dependent manner. The term is meant to encompass prokaryotic mutL proteins as well as homologs, orthologs, paralogs, variants, or fragments thereof. MutL proteins include, for example, polypeptides encoded by nucleic acids having the following GenBank accession Nos. AF170912 (C. crescentus), AI518690 (D. melanogaster), AI456947 (D. melanogaster), AI389544 (D. melanogaster), AI387992 (D. melanogaster), AI292490 (D. melanogaster), AF068271 (D. melanogaster), AF068257 (D. melanogaster), U50453 (T. aquaticus), U27343 (B. subtilis), U71053 (U71053 (T. maritima), U71052 (A. pyrophilus), U13696 (H. sapiens), U13695 (H. sapiens), M29687 (S. typhimurium), M63655 (E. coli) and L19346 (E. coli). MutL homologs include, for example, eukaryotic MLH1, MLH2, PMS1, and PMS2 proteins (see e.g., U.S. Pat. Nos. 5,858,754 and 6,333,153, incorporated herein by reference in their entirety).

As used herein, the term “mutS” refers to a DNA-mismatch binding protein that recognizes and binds to a variety of mispaired bases and small (1-5 bases) single-stranded loops. The term is meant to encompass prokaryotic mutS proteins as well as homologs, orthologs, paralogs, variants, or fragments thereof. The term also encompasses homo- and hetero-dimmers and multimers of various mutS proteins. MutS proteins include, for example, polypeptides encoded by nucleic acids having the following GenBank accession Nos. AF146227 (M. musculus), AF193018 (A. thaliana), AF144608 (V. parahaemolyticus), AF034759 (H. sapiens), AF104243 (H. sapiens), AF007553 (T. aquaticus caldophilus), AF109905 (M. musculus), AF070079 (H. sapiens), AF070071 (H. sapiens), AH006902 (H. sapiens), AF048991 (H. sapiens), AF048986 (H. sapiens), U33117 (T. aquaticus), U16152 (Y. enterocolitica), AF000945 (V. cholarae), U698873 (E. coli), AF003252 (H. influenzae strain b (Eagan)), AF003005 (A. thaliana), AF002706 (A. thaliana), L10319 (M. musculus), D63810 (T. thermophilus), U27343 (B. subtilis), U71155 (T. maritima), U71154 (A. pyrophilus), U16303 (S. typhimurium), U21011 (M. musculus), M84170 (S. cerevisiae), M84169 (S. cerevisiae), M18965 (S. typhimurium) and M63007 (A. vinelandii). MutS homologs include, for example, eukaryotic MSH2, MSH3, MSH4, MSH5, and MSH6 proteins (see e.g., U.S. Pat. Nos. 5,858,754 and 6,333,153).

As used herein, the terms “nucleic acid molecule,” “nucleic acid sequence,” “nucleic acid fragment,” “oligonucleotide” and “polynucleotide” are used interchangeably and are intended to include, but not limited to, a polymeric form of nucleotides that may have various lengths, either deoxyribonucleotides or ribonucleotides, or analogs thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, ribozymes, small interfering RNA (siRNA), miRNA, small nucleolar RNA (snoRNA), cDNA, recombinant polynucleotides, branched polynucleotides, plasmids, vectors, isolated DNA of a sequence, isolated RNA of a sequence, nucleic acid probes, and primers. Oligonucleotides useful in the methods described herein may comprise natural nucleic acid sequences and variants thereof, artificial nucleic acid sequences, or a combination of such sequences.

The terms “oligonucleotide” or “polynucleotide,” which are used synonymously, are intended to refer to a polymer of natural or modified nucleosidic monomers linked by phosphodiester bonds or analogs thereof. The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. Oligonucleotides and/or polynucleotides comprising probes or primers usually have lengths in the range of from 8 to 60 nucleotides, and more usually, from 8 to 25 or about 10 nucleotides. Substrate oligonucleotides and/or polynucleotides usually have lengths in the range of from 20 to 250 nucleotides, and more usually, from 50 to 200 or about 60 nucleotides.

Oligonucleotides and polynucleotides may be natural or synthetic. Oligonucleotides and polynucleotides include deoxyribonucleosides, ribonucleosides, and non-natural analogs thereof, such as anomeric forms thereof, peptide nucleic acids (PNAs), and the like, provided that they are capable of specifically binding to a target genome by way of a regular pattern of monomer-to-monomer interactions, such as Watson-Crick type of base pairing, base stacking, Hoogsteen or reverse Hoogsteen types of base pairing, or the like. Non-limiting examples of oligonucleotides and polynucleotides include a gene, a gene fragment, an exon, an intron, intergenic DNA (including, without limitation, heterochromatic DNA), messenger RNA (mRNA), transfer RNA, ribosomal RNA, ribozymes, small interfering RNA (siRNA), miRNA, small nucleolar RNA (snoRNA), cDNA, recombinant polynucleotides, branched polynucleotides, plasmids, vectors, isolated DNA of a sequence, isolated RNA of a sequence, nucleic acid probes, and primers. Oligonucleotides and polynucleotides useful in the methods described herein may comprise natural nucleic acid sequences and variants thereof, artificial nucleic acid sequences, or a combination of such sequences.

A polynucleotide and/or oligonucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides and/or oligonucleotide may optionally include one or more non-standard nucleotide(s), nucleotide analog(s) and/or modified nucleotides.

Examples of modified nucleotides include, but are not limited to diaminopurine, S²T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone.

Oligonucleotide and/or polynucleotide sequences may be isolated from natural sources or purchased from commercial sources. Oligonucleotide and/or polynucleotide sequences may also be prepared by any suitable method, e.g., standard phosphoramidite methods such as those described by Beaucage and Carruthers ((1981) Tetrahedron Lett. 22: 1859) or the triester method according to Matteucci et al. (1981) J. Am. Chem. Soc. 103:3185), or by other chemical methods using either a commercial automated oligonucleotide synthesizer or high-throughput, high-density array methods known in the art (see U.S. Pat. Nos. 5,602,244, 5,574,146, 5,554,744, 5,428,148, 5,264,566, 5,141,813, 5,959,463, 4,861,571 and 4,659,774, incorporated herein by reference in its entirety for all purposes). Pre-synthesized oligonucleotides may also be obtained commercially from a variety of vendors.

In certain exemplary embodiments, oligonucleotide sequences may be prepared using a variety of microarray technologies known in the art. Pre-synthesized oligonucleotide and/or polynucleotide sequences may be attached to a support or synthesized in situ using light-directed methods, flow channel and spotting methods, inkjet methods, pin-based methods and bead-based methods set forth in the following references: McGall et al. (1996) Proc. Natl. Acad. Sci. U.S.A. 93:13555; Synthetic DNA Arrays In Genetic Engineering, Vol. 20:111, Plenum Press (1998); Duggan et al. (1999) Nat. Genet. S21:10; Microarrays: Making Them and Using Them In Microarray Bioinformatics, Cambridge University Press, 2003; U.S. Patent Application Publication Nos. 2003/0068633 and 2002/0081582; U.S. Pat. Nos. 6,833,450, 6,830,890, 6,824,866, 6,800,439, 6,375,903 and 5,700,637; and PCT Application Nos. WO 04/031399, WO 04/031351, WO 04/029586, WO 03/100012, WO 03/066212, WO 03/065038, WO 03/064699, WO 03/064027, WO 03/064026, WO 03/046223, WO 03/040410 and WO 02/24597.

In certain exemplary embodiments, a detectable label can be used to detect one or more oligonucleotides and/or polynucleotides described herein. Examples of detectable markers include various radioactive moieties, enzymes, prosthetic groups, fluorescent markers, luminescent markers, bioluminescent markers, metal particles, protein-protein binding pairs, protein-antibody binding pairs and the like. Examples of fluorescent proteins include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin and the like. Examples of bioluminescent markers include, but are not limited to, luciferase (e.g., bacterial, firefly, click beetle and the like), luciferin, aequorin and the like. Examples of enzyme systems having visually detectable signals include, but are not limited to, galactosidases, glucorinidases, phosphatases, peroxidases, cholinesterases and the like. Identifiable markers also include radioactive compounds such as ¹²⁵I, ³⁵S, ¹⁴C, or ³H. Identifiable markers are commercially available from a variety of sources.

Fluorescent labels and their attachment to nucleotides and/or oligonucleotides are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). Particular methodologies applicable to the invention are disclosed in the following sample of references: U.S. Pat. Nos. 4,757,141, 5,151,507 and 5,091,519. In one aspect, one or more fluorescent dyes are used as labels for labeled target sequences, e.g., as disclosed by U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); Lee et al.; U.S. Pat. No. 5,066,580 (xanthine dyes); U.S. Pat. No. 5,688,648 (energy transfer dyes); and the like. Labelling can also be carried out with quantum dots, as disclosed in the following patents and patent publications: U.S. Pat. Nos. 6,322,901, 6,576,291, 6,423,551, 6,251,303, 6,319,426, 6,426,513, 6,444,143, 5,990,479, 6,207,392, 2002/0045045 and 2003/0017264. As used herein, the term “fluorescent label” includes a signaling moiety that conveys information through the fluorescent absorption and/or emission properties of one or more molecules. Such fluorescent properties include fluorescence intensity, fluorescence lifetime, emission spectrum characteristics, energy transfer, and the like.

Commercially available fluorescent nucleotide analogues readily incorporated into nucleotide and/or oligonucleotide sequences include, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY TM 630/650-14-dUTP, BODIPY TM 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY TM FL-14-UTP, BODIPY TMR-14-UTP, BODIPY TM TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.) and the like. Protocols are known in the art for custom synthesis of nucleotides having other fluorophores (See, Henegariu et al. (2000) Nature Biotechnol. 18:345).

Other fluorophores available for post-synthetic attachment include, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, Cy7 (Amersham Biosciences, Piscataway, N.J.) and the like. FRET tandem fluorophores may also be used, including, but not limited to, PerCP-Cy5.5, PE-Cy5, PE-Cy5.5, PE-Cy7, PE-Texas Red, APC-Cy7, PE-Alexa dyes (610, 647, 680), APC-Alexa dyes and the like.

Metallic silver or gold particles may be used to enhance signal from fluorescently labeled nucleotide and/or oligonucleotide sequences (Lakowicz et al. (2003) BioTechniques 34:62).

Biotin, or a derivative thereof, may also be used as a label on an oligonucleotide sequence, and subsequently bound by a detectably labeled avidin/streptavidin derivative (e.g. phycoerythrin-conjugated streptavidin), or a detectably labeled anti-biotin antibody. Digoxigenin may be incorporated as a label and subsequently bound by a detectably labeled anti-digoxigenin antibody (e.g. fluoresceinated anti-digoxigenin). An aminoallyl-dUTP residue may be incorporated into an oligonucleotide sequence and subsequently coupled to an N-hydroxy succinimide (NHS) derivatized fluorescent dye. In general, any member of a conjugate pair may be incorporated into a detection oligonucleotide provided that a detectably labeled conjugate partner can be bound to permit detection. As used herein, the term antibody refers to an antibody molecule of any class, or any sub-fragment thereof, such as an Fab.

Other suitable labels for an oligonucleotide and/or polynucleotide sequence may include fluorescein (FAM), digoxigenin, dinitrophenol (DNP), dansyl, biotin, bromodeoxyuridine (BrdU), hexahistidine (6× His), phosphor-amino acids (e.g. P-tyr, P-ser, P-thr) and the like. In one embodiment the following hapten/antibody pairs are used for detection, in which each of the antibodies is derivatized with a detectable label: biotin/α-biotin, digoxigenin/α-digoxigenin, dinitrophenol (DNP)/α-DNP, 5-Carboxyfluorescein (FAM)/α-FAM.

Oligonucleotide and/or polynucleotide sequences can be indirectly labeled, especially with a hapten that is then bound by a capture agent, e.g., as disclosed in Holtke et al., U.S. Pat. Nos. 5,344,757; 5,702,888; and 5,354,657; Huber et al., U.S. Pat. No. 5,198,537; Miyoshi, U.S. Pat. No. 4,849,336; Misiura and Gait, PCT publication WO 91/17160; and the like. Many different hapten-capture agent pairs are available for use with the invention, either with a target sequence or with a detection oligonucleotide used with a target sequence, as described below. Exemplary, haptens include, biotin, des-biotin and other derivatives, dinitrophenol, dansyl, fluorescein, CY5, and other dyes, digoxigenin, and the like. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In certain exemplary embodiments, a first oligonucleotide (e.g., substrate oligonucleotide and/or polynucleotide) sequence is annealed to a second oligonucleotide (e.g., primer and/or substrate oligonucleotide) sequence. The terms “annealing” and “hybridization,” as used herein, are used interchangeably to mean the formation of a stable duplex. In one aspect, stable duplex means that a duplex structure is not destroyed by a stringent wash, e.g., conditions including temperature of about 5° C. less that the Tₘ of a strand of the duplex and low monovalent salt concentration, e.g., less than 0.2 M, or less than 0.1 M. The term “perfectly matched,” when used in reference to a duplex means that the polynucleotide and/or oligonucleotide strands making up the duplex form a double stranded structure with one another such that every nucleotide in each strand undergoes Watson-Crick base pairing with a nucleotide in the other strand. The term “duplex” includes, but is not limited to, the pairing of nucleoside analogs, such as deoxyinosine, nucleosides with 2-aminopurine bases, PNAs, and the like, that may be employed. A “mismatch” in a duplex between two oligonucleotides means that a pair of nucleotides in the duplex fails to undergo Watson-Crick bonding.

As used herein, the term “hybridization conditions,” will typically include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. Hybridizations are usually performed under stringent conditions, i.e., conditions under which a probe will specifically hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Generally, stringent conditions are selected to be about 5° C. lower than the Tₘ for the specific sequence at a defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM Na phosphate, 5 mM EDTA, pH 7.4) and a temperature of 25-30° C. are suitable for allele-specific probe hybridizations. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1^{st} Ed., BIOS Scientific Publishers Limited (1999). As used herein, the terms “hybridizing specifically to” or “specifically hybridizing to” or similar terms refer to the binding, duplexing, or hybridizing of a molecule substantially to a particular nucleotide sequence or sequences under stringent conditions.

The contents of all references, patents and published patent applications cited throughout this application are hereby incorporated by reference in their entirety for all purposes. It is to be understood that the embodiments of the present invention which have been described are merely illustrative of some of the applications of the principles of the present invention. Numerous modifications may be made by those skilled in the art based upon the teachings presented herein without departing from the true spirit and scope of the invention.

What is claimed is:

1. A method of making a polynucleotide comprising the steps of:
a) providing an oligonucleotide array having a plurality of adjacent, discrete features attached thereto, wherein each feature comprises a substrate oligonucleotide;
b) contacting a first discrete feature having a first substrate attached thereto with an oligonucleotide primer;
c) allowing the oligonucleotide primer to hybridize to the first substrate oligonucleotide and extending the substrate oligonucleotide to generate an extended oligonucleotide;
d) releasing the extended oligonucleotide and allowing the extended oligonucleotide to contact an adjacent, second discrete feature having a second substrate attached thereto; and
e) allowing the extended oligonucleotide to hybridize to the second substrate oligonucleotide and extending the hybridized extended oligonucleotide and second substrate oligonucleotide to generate a first polynucleotide.

2. The method of claim 1, wherein the step of releasing is performed by contacting the extended oligonucleotide with a helicase, a strand displacement polymerase or heat.

3. The method of claim 1, wherein the oligonucleotide array comprises a chip, a slide or a plate.

4. The method of claim 1, wherein a pair of primers is provided in step a).

5. The method of claim 1, wherein contact occurs by diffusion.

6. The method of claim 1, wherein the first polynucleotide is amplified.

7. The method of claim 6, wherein amplification is performed by polymerase chain reaction or ligase chain reaction.

8. The method of claim 1, further comprising removing one or both of an extended oligonucleotide and a first polynucleotide having a mismatch.

9. The method of claim 8, wherein the one or both of the extended oligonucleotide and the first polynucleotide having a mismatch are removed by mismatch-sensitive hybridization, mutS binding, MutHSL cleavage near the mismatch or cleavage at the mismatch.

10. The method of claim 1, wherein the oligonucleotide primer is between 8 and 25 nucleotides in length.

11. The method of claim 1, wherein the first and second substrate oligonucleotides are between 50 and 100 nucleotides in length.

12. The method of claim 1, wherein the first polynucleotide is greater than 100 nucleotides in length.

13. The method of claim 1, wherein the first polynucleotide is between 100 and 150 nucleotides in length.

14. The method of claim 1, wherein the primer is added by ink-jet printing.

15. The method of claim 1, further comprising the steps of:
f) releasing the first polynucleotide and allowing the first polynucleotide to contact an adjacent, third discrete feature having a third substrate attached thereto; and
g) allowing the first polynucleotide to hybridize to the third substrate oligonucleotide and extending the hybridized first polynucleotide and third substrate oligonucleotide to generate a second polynucleotide.

16. The method of claim 15, wherein the second polynucleotide is greater than 200 nucleotides in length.

17. The method of claim 15, wherein the second polynucleotide is between 200 and 300 nucleotides in length.
